(19) United States
(12) Patent Application Publication
(10) Pub. No.: US 2013/0011844 A1
(43) Pub. Date: Jan. 10, 2013

(54) Novel Biomarker Indicative of Ischemic Brain Injury and Its Use

(75) Inventors: Yun-Bae Kim, Daejeon (KR); Seong Soo Joo, Suwon-si (KR); Dong Sun Park, Cheongju-si (KR)

(73) Assignees: Kangnung-Wonju National University Industry Academy Cooperation Group, Gangneung City (KR); Chungbuk National University Industry Academic Cooperation Foundation, Chungbuk (KR)

(21) Appl. No.: 13/176,060

(22) Filed: Jul. 5, 2011

(51) Int. Cl. C12Q 1/68; G01N 33/566

(52) U.S. Cl. 435/6.12; 435/7.92

(57) ABSTRACT

The present invention relates to a method for detecting neuronal injury in a mammalian subject. The biomarker of this invention specifically increases in serum of the mammalian that has neuronal injuries. In addition, the biomarker of this invention permits to identify and predict neuronal injuries.

### BACKGROUND OF THE INVENTION

1. Field of the Invention

The present invention relates to a novel biomarker indicative of ischemic brain injury and their uses.

2. Description of the Related Art

During the last few decades, researchers have attempted to identify potential neurobiochemical markers of brain injury in the body fluids, since imaging techniques, such as computed tomography, have a limited capacity to identify lesions during the first crucial hours without massive tissue destruction [1]. Several studies have demonstrated a significant association between clinical deficit or infarct volume and the release of S100B, glial fibrillary acidic protein (GFAP) or neuron-specific enolase (NSE) into the peripheral blood in stroke patients, indicating that the serum levels of these proteins can be readily used as surrogate markers to assess the outcome of a stroke [2-4]. However, the S100B protein is derived not only from neural tissue, but also peripheral cells including tumors [5-8]. Also the correlation between blood NSE levels and infarction volume [4] was found to be controversial in clinical trials [9, 10]. Furthermore, significant increases in the blood level of NSE and GFAP were observed 2-3 days after acute stroke [3, 11, 12], while S100B was detected in only patients with large infarction or intracerebral hemorrhage [13], leading to low diagnostic accuracy [14].

Clinically, tissue-type plasminogen activator (t-PA) has been used for the emergency treatment of acute ischemic stroke [15]. However, t-PA treatment remains problematic because of its extremely limited window of efficacy, which should be administered within 3 hours after the onset of the stroke, and potential hemorrhagic side effect which could lead to a fatal outcome [16]. In this context, a more sensitive early marker is needed for monitoring and evaluating the use of therapeutic interventions such as neuroprotective drugs or t-PA, which is expected to be a major part of future treatments.

Ideally, these biomarkers should indicate the amount of tissue damage early enough and be derived from the major target tissue of ischemia. Microtubule associated protein 2 (MAP2) is abundantly expressed in the soma and dendrites of neuronal cells [17]. Alterations in MAP2 expression are of key importance in differentiation, growth, plasticity, and even degeneration of neurons [18]. Immunohistochemical studies showed that MAP2 disappeared in damaged brain regions following ischemia and hypoxia [19-22]. Hypoxia and hyperosmotic mannitol disrupt the blood-brain barrier (BBB). This disruption may release CNS proteins such as α₂-macroglobulin and S100B [23, 24]. In the present invention, we measured the release of MAP2 and GFAP into the cerebrospinal fluid (CSF) and blood after inducing cerebral ischemia in a stroke model to determine whether MAP2 could be used as an ideal early marker for assessing the severity of tissue destruction.

Throughout this application, various patents and publications are referenced, and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive studies to develop a novel biomarker for identifying neuronal injuries at a molecular level in a high-throughput and accurate manner. As results, we have discovered a biomarker capable of early detecting and predicting neuronal injuries.

Accordingly, it is an object of this invention to provide a method for detecting neuronal injuries in a mammalian subject.

Other objects and advantages of the present invention will become apparent from the detailed description to follow taken in conjugation with the appended claims and drawings.

### BRIEF DESCRIPTION OF THE DRAWINGS

The application file contains drawings executed in color (FIGS. 1 and 2). Copies of this patent or patent application with color drawings will be provided by the Office upon request and payment of the necessary fee.

FIG. 1 shows representative section of ischemic rat brains. Two mm coronal sections were stained with 2% 2,3,5-triphenyltetrazolium chloride (TTC) after middle cerebral artery occlusion for the times indicated (upper). The total infarction volume (%) was calculated from the TTC-stained brain sections (lower).

FIG. 2 represents schematic presentation of section of ischemia/reperfused rat brains. Brain sections were stained with 2% 2,3,5-triphenyltetrazolium chloride (TTC), 24 hours after reperfusion following ischemia for the times indicated (upper). The total infarction volume (%) was calculated from the TTC-stained brain sections (lower).

FIG. 3 represents expression level of GFAP (glial fibrillary acidic protein) and MAP2 (microtubule-associated protein 2) in the CSF(cerebrospinal fluid) and serum. Western blot analysis was performed 24 and 48 hours after reperfusion following 3-hour ischemia.

FIG. 4 represents expression level of GFAP and MAP2 in the serum. Western blot analysis was performed immediately before (0 hour) and 2 hours after reperfusion following 0.5-3 hour ischemia. *P<0.05 vs. non-ischemic control.

FIG. 5 represents time-course of the expression level of MAP2 in the serum. Western blot analysis was performed at each time points after reperfusion following 3-hour ischemia.

FIG. 6 shows western blot analysis of MAP2 in the brain and serum. (A) Normal and ischemia/reperfused brain proteins after reperfusion following 3-hour ischemia were prepared and a protein separation was performed in 10% and 7.5% SDS-PAGE to clarify high-molecular weight- and low-molecular weight-MAP2. Lane 1, 40 μg; Lane 2, 20 μg. (B) Sera from normal and ischemia/reperfused rats identical to (A) were separated on a 10% SDS-PAGE.

### DETAILED DESCRIPTION OF THIS INVENTION

In an aspect of this invention, there is provided a method for detecting neuronal injury in a mammalian subject, comprising:
(a) providing a biological sample from the mammalian subject; and
(b) detecting the level of a MAP2 (microtubule-associated protein 2) protein or a MAP2 nucleic acid in the biological sample, relative to the level of the MAP2 protein or the MAP2 nucleic acid in a control sample from a normal mammalian, wherein an increased level of the MAP2 protein or the MAP2 nucleic acid in the biological sample compared to the control sample indicates that the mammalian has the neuronal injury.

The present inventors have made intensive studies to develop a novel biomarker for identifying neuronal injury at a molecular level in a high-throughput and accurate manner. As results, we have discovered a biomarker capable of early detecting and predicting neuronal injuries.

According to a preferred embodiment, the expression level of MAP2 specifically increases in serum of the mammalian that has neuronal injuries.

The term used herein “neuronal injury” includes neurological insults (such as ischemia as a result of stroke, cardiac arrest, hypoglycemia, epilepsy or trauma) and neurodegenerative disorder (such as Huntington's disease, Alzheimer's disease and amyotropic lateral sclerosis), preferably ischemic neurological insult.

The ischemic neurological insult is caused by ischemia. Ischemia is the lack of oxygen supply to the cells. In animals, including humans, the underlying cause of ischemia is typically a cardiovascular disease, where blood vessels may be affected by arteriosclerosis. Cardiac ischemia is caused by restriction of blood flow in the coronary arteries, e.g. due to atherosclerosis. This reduced blood flow and the resulting lack of oxygen to the myocytes in the heart may lead to several effects, including hypokinesia, dyskinesia, akinesia and hibernating cells. These various effects may in turn decrease the hemodynamic performance of the heart, which ultimately can cause cardiac asynchrony, worsening heart failure and further decrease in pumping capacity.

The biological sample used in this invention may include tissue, cell, blood, serum, plasma, saliva, cerebrospinal fluid and urine, preferably blood, serum and plasma, more preferably serum.

The biological sample is obtained from mammalian subjects including human, mouse, rat, cow, horse, pig and cattle. Preferably, the biological sample is obtained from human, more preferably human risk of neuronal injury or suffering from neuronal injury.

The detection of the expression level of MAP2 may be carried out in either a protein or a nucleic acid level.

Where the MAP2 protein is detected for detecting neuronal injuries, it is preferred that the detection of the step (b) is carried out by an immunoassay, i.e. antigen-antibody reactions. The immunoassay may be performed using an antibody or aptamer binding specifically to the MAP2 protein.

The antibody against the biomarker used in this invention may polyclonal or monoclonal, preferably monoclonal. The antibody could be prepared according to conventional techniques such as a fusion method (Kohler and Milstein, European Journal of Immunology, 6: 511-519(1976)), a recombinant DNA method (U.S. Pat. No. 4,816,56) or a phage antibody library (Clackson, et al., Nature, 352: 624-628 (1991) and Marks, et al., J. Mol. Biol., 222: 58, 1-597 (1991)). The general procedures for antibody production are described in Harlow, E. and Lane, D., Antibodies: A Laboratory Manual, Cold Spring Harbor Press, New York, 1988; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, N.Y., 1991, which are incorporated herein by references. For example, the preparation of hybridoma cell lines for monoclonal antibody production is done by fusion of an immortal cell line and the antibody-producing lymphocytes. This can be done by techniques well known in the art. Polyclonal antibodies may be prepared by injection of the protein antigen to suitable animal, collecting antiserum containing antibodies from the animal, and isolating specific antibodies by any of the known affinity techniques.

Where the method of this invention is performed using antibodies or aptamers to the biomarker protein, it could be carried out according to conventional immunoassay procedures for identifying neuronal injuries.

Such immunoassay may be executed by quantitative or qualitative immunoassay protocols, including radioimmunoassay, radioimmuno-precipitation, immunoprecipitation, immunostaining assay, enzyme-linked immunosorbent assay (ELISA), capture-ELISA, inhibition or competition assay, sandwich assay, flow cytometry, immunofluorescence assay and immuoaffinity assay, but not limited to. The immunoassay and immuostaining procedures can be found in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies A Laboratory Manual, Cold Spring Harbor Press, 1999, which are incorporated herein by references.

For example, according to the radioimmunoassay method, the radioisotope (e.g., C¹⁴, I¹²⁵, P³² and S³⁵)-labeled antibody may be used to detect the biomarker protein of this invention.

In addition, according to the ELISA method, the illustrative example of the present invention may comprise the steps of: (i) coating a surface of solid substrates with cell lysates to be analyzed; (ii) incubating the coated cell lysates with a primary antibody against a biomarker protein; (iii) incubating the resultant of step (ii) with a secondary antibody conjugated with an enzyme; and (iv) measuring the activity of the enzyme.

The solid substrate useful in this invention includes carbohydrate polymer (e.g., polystyrene and polypropylene), glass, metal or gel, and most preferably microtiter plates.

The enzyme conjugated with the secondary antibody includes an enzyme which catalyzes colorimetric, fluorometric, luminescence or infra-red reactions, e.g., including alkaline phosphatase, β-galactosidase, horseradish peroxidase, luciferase and cytochrome P₄₅₀, but not limited to. Where using alkaline phosphatase, bromochioroindolylphosphate (BCIP), nitro blue tetrazolium (NBT), naphthol-AS-B1-phosphate and enhanced chemifluorescence (ECF) may be used as a substrate for color-developing reactions; in the case of using horseradish peroxidase, chloronaphtol, aminoethylcarbazol, diaminobenzidine, D-luciferin, lucigenin(bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) and naphtol/pyronin, glucose oxidase and tNBT (nitroblue tetrazolium) and m-PMS (phenzaine methosulfate) may be used as a substrate.

According to the capture-ELISA method, the illustrative example of the present method may comprise the steps of: (i) coating a surface of a solid substrate with an antibody of a biomarker protein as a capturing antibody; (ii) incubating the capturing antibody with a cell sample; (iii) incubating the resultant of step (ii) with a detecting antibody having a fluorescent label which reacts with the biomarker protein specifically; and (iv) measuring the signal generated from the label.

The detecting antibody includes a substance generating a detectable signal. The signal-generating substance bound to antibody includes, but is not limited to, chemical (e.g., biotin), enzyme (alkaline phosphatase, β-galactosidase, horseradish peroxidase and cytochrome P₄₅₀), radio-isotope (e.g., C¹⁴, I¹²⁵, P³² and S³⁵), fluorescent (e.g., fluorescein), luminescent, chemiluminescent and FRET (fluorescence resonance energy transfer) substances. Various methods for labels and labelings are described in Ed Harlow and David Lane, Using Antibodies:A Laboratory Manual, Cold Spring Harbor Laboratory Press, 1999.

The analysis for measuring the activity or the signal of final enzyme in the ELISA and capture-ELISA method may be carried out by various methods known to those skilled in the art. The signal detection allows for a qualitative or quantitative analysis of the present marker. For example, the signal of each biotin- and luciferase-labeled protein may be feasibly detected using streptavidin and luciferin.

Alternatively, aptamer having a specific binding affinity to the biomarker of the present invention may be used instead of antibody. Aptamer means an oligonucleic acid or peptide molecule, and general descriptions of aptamer are disclosed in Bock L C et al., Nature 355 (6360): 564-566 (1992); Hoppe-Seyler F, Butz K “Peptide aptamers: powerful new tools for molecular medicine”. J Mol Med. 78 (8): 426-430 (2000); and Cohen B A, Colas P, Brent R. “An artificial cell-cycle inhibitor isolated from a combinatorial library”. Proc Natl Acad Sci USA. 95 (24): 14272-14277 (1998).

The final signal intensity measured by the above-mentioned immunoassay procedures is indicative of neuronal injuries. When the signal to the biomarker of this invention in a sample of interest is stronger than that in normal samples, the sample is determined to have neuronal injuries.

The biomarker of the present invention is a biomolecule expressed highly in neuronal injury. The high expression of the biomarker may be measured at mRNA or protein level. The term “high expression” with reference to neuronal injury means that the nucleotide sequence of interest in a sample to be analyzed is much more highly expressed than that in the normal sample, for instance, a case analyzed as high expression according to analysis methods known to those skilled in the art, e.g., RT-PCR method or ELISA method (See, Sambrook, J., et al., Molecular Cloning. A Laboratory Manual, 3rd ed. Cold Spring Harbor Press (2001)). Using analysis methods as described above, where the biomarker of the present invention is much more highly expressed 2-10 folds in neuronal injured cells than in normal cells, this case is determined as “high expression” and identified as neuronal injury in the present invention.

The present invention may be generally carried out by nucleic acid amplifications using mRNA molecules in samples as templates and primers to be annealed to mRNA or cDNA.

For obtaining mRNA molecules, total RNA is isolated from samples. The isolation of total RNA may be performed by various methods (Sambrook, J. et al., Molecular Cloning. A Laboratory Manual, 3rd ed. Cold Spring Harbor Press (2001); Tesniere, C. et al, Plant Mol. Biol. Rep., 9: 242 (1991); Ausubel, F. M. et al., Current Protocols in Molecular Biology, John Willey & Sons (1987); and Chomczynski, P. et al., Anal. Biochem. 162: 156 (1987)). For example, total RNA in cells may be isolated using Trizol. Afterwards, cDNA molecules are synthesized using mRNA molecules isolated and then amplified. Since total RNA molecules used in the present invention are isolated from human samples, mRNA molecules have poly-A tails and converted to cDNA by use of dT primer and reverse transcriptase (PNAS USA, 85: 8998 (1988); Libert F, et al., Science, 244: 569 (1989); and Sambrook, J. et al., Molecular Cloning. A Laboratory Manual, 3rd ed. Cold Spring Harbor Press (2001)). cDNA molecules synthesized are then amplified by amplification reactions.

The primers used for the present invention is hybridized or annealed to a region on template so that double-stranded structure is formed. Conditions of nucleic acid hybridization suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the amplification step of the present methods, which includes “Klenow” fragment of E. coli DNA polymerase I, a thermostable DNA polymerase and bacteriophage T7 DNA polymerase. Preferably, the polymerase is a thermostable DNA polymerase obtained from a variety of bacterial species, including Thermus aquaticus (Taq), Thermus thermophilus (Tth), Thermus filiformis, Thermis flavus, Thermococcus literalis, and Pyrococcus furiosus (Pfu).

When a polymerization reaction is being conducted, it is preferable to provide the components required for such reaction in excess in the reaction vessel. Excess in reference to components of the amplification reaction refers to an amount of each component such that the ability to achieve the desired amplification is not substantially limited by the concentration of that component. It is desirable to provide to the reaction mixture an amount of required cofactors such as Mg²⁺, and dATP, dCTP, dGTP and dTTP in sufficient quantity to support the degree of amplification desired. All of the enzymes used in this amplification reaction may be active under the same reaction conditions. Indeed, buffers exist in which all enzymes are near their optimal reaction conditions. Therefore, the amplification process of the present invention can be done in a single reaction volume without any change of conditions such as addition of reactants.

Annealing or hybridization in the present invention is performed under stringent conditions that allow for specific binding between the target nucleotide sequence and the primer. Such stringent conditions for annealing will be sequence-dependent and varied depending on environmental parameters.

The amplified cDNA to the nucleotide sequence of the biomarker of this invention are then analyzed to assess their expression level using suitable methods. For example, the amplified products are resolved by a gel electrophoresis and the bands generated are analyzed to assess the expression level of the nucleotide sequence of the present biomarker. When the expression level of the nucleotide sequence of the present biomarker from a sample to be diagnosed is measured to be higher than normal samples (normal cells), the sample can be determined to have neuronal injury.

According to a preferred embodiment, the detection of the step (b) is carried out before the appearance of visible lesions in neuronal tissues. It is one of strikingly prominent advantages of the MAP2 biomarker that MAP2 is detected in an earlier stage of neuronal injuries. MAP2 can be detected before the appearance of visible lesions of neuronal injuries. The term “lesion” used herein means any abnormal tissue found on or in an organism. Lesions are caused by any process that damages tissues.

More preferably, the detection of the step (b) is carried out within 10-60 min (still more preferably 10-50 min, still further more preferably 20-40 min) of the neurological insult.

In the present invention, the present inventors provide a plausible marker for neuronal injuries. As demonstrated in Examples herein below, MAP2 may be observed in a significant increase level as early as 0.5 hour in ischemic animals having no visible lesions such as infarction. Interestingly, a more prominent increase in MAP2 may be achieved within 2 hours in 0.5-1-hour ischemia/reperfused rats, which later displayed little or no infarction.

The early microlesions in neurons can be detected by analyzing the level of MAP2 in a variety of biological samples such as the blood, which precede the appearance of visible lesions. Therefore, we propose that MAP2, which shows a high level in biological samples (e.g. the blood), can be used as an early sensitive index of neuronal injuries such as neurotoxic insult, providing patients with information on the severity and prognosis of CNS (central nervous system) damage including brain damage for emergency or follow-up treatment.

The present invention will now be described in further detail by examples. It would be obvious to those skilled in the art that these examples are intended to be more concretely illustrative and the scope of the present invention as set forth in the appended claims is not limited to or by the examples.

### EXAMPLE

### Material and Methods

### Animals

Nine-week-old male Sprague-Dawley rats (300-330 g) were purchased from a commercial breeder (Samtaco, Osan, Korea). They were housed in an environmentally controlled room that was held at a constant temperature (23±3° C.), relative humidity (50±10%), and had a 12-hour light cycle. Animals were fed with standard rodent chow and purified water ad libitum.

### Middle Cerebral Artery Occlusion (MCAO) Surgery

The focal cerebral ischemia was produced using a modified monofilament method as previously described [25]. Briefly, rats were anesthetized with 5% isoflurane in 25% )₂/75% N₂ for induction, and then maintained with 1.5-2% isoflurane. The rectal temperature was maintained at 37-38° C. The left common carotid artery was exposed, and a 25-mm nylon monofilament that was coated with silicon (0.3 mm in diameter) was inserted from the external carotid artery into the lumen of the internal carotid artery until the tip occluded the origin of the middle cerebral artery. After closure of the operative sites, the animals were allowed to wake from the anesthesia. In ischemia groups, the occlusion was maintained for 0.5, 1, 2, 3, 6, 12 or 24 hours, while in ischemia-reperfusion groups, the filament was withdrawn after 0.5, 1, 2 or 3-hour ischemia.

### Infarct Volume

Two mm-thick coronal sections of the brain were cut using a pre-cooled matrix, and stained with 2% 2,3,5-triphenyltetrazolium chloride (TTC) solution in saline for 15 min at 37° C. under gentle shaking [26]. The TTC-stained slices were fixed overnight in 10% neural buffered formalin. The infarction area of each section was determined using a computerized image analysis system (Imageinside, Daejeon, Korea), and the areas from all sections were quantified and multiplied by their thickness to obtain the total infarct volume.

### Western Blot Analysis

At different time points after ischemia and/or reperfusion, CSF and blood was collected to analyze potential candidate marker proteins. Serum was obtained by centrifugation at 3,000 rpm for 20 min at 4° C. CSF and serum proteins were denatured by boiling for 5 minutes at 95° C. in 0.5 M Tris-HCl buffer (pH 6.8) that contained 10% sodium dodecyl sulfate (SDS) and 10% ammonium persulfate (APS), separated by electrophoresis on a 7.5% SDS-polyacrylamide gel (SDS-PAGE) and transferred onto a polyvinylidene difluoride membrane (PVDF) in 25 mM Tris buffer that contained 15% methanol, 1% SDS and 192 mM glycine. After blocking for 2 hours with 5% skim milk in Tris-buffered saline Tween (TBS-T, 20 mM Tris pH 7.6, 137 mM NaCl, 0.1% Tween 20), the membrane was incubated with an anti-rabbit MAP2 monoclonal antibody (Santa Cruz Biotechnology, San Jose, Calif., USA) for 1.5 hours at room temperature. After washing with TBS-T, the membrane was incubated with a secondary goat anti-rabbit IgG conjugated with horseradish peroxidase (Zymed, San Francisco, Calif., USA), for 1 hour at room temperature. The membrane was then developed using an ECL solution (Amersham Biosciences, Sunnyvale, Calif., USA).

### Statistical Analysis

Data are presented as means±SEM. The statistical significance between group comparisons for MAP2 was determined by one-way analysis of variance (ANOVA). P<0.05 was considered to be statistically significant.

### Results

### Effect of Ischemic Duration on Infarction Volume

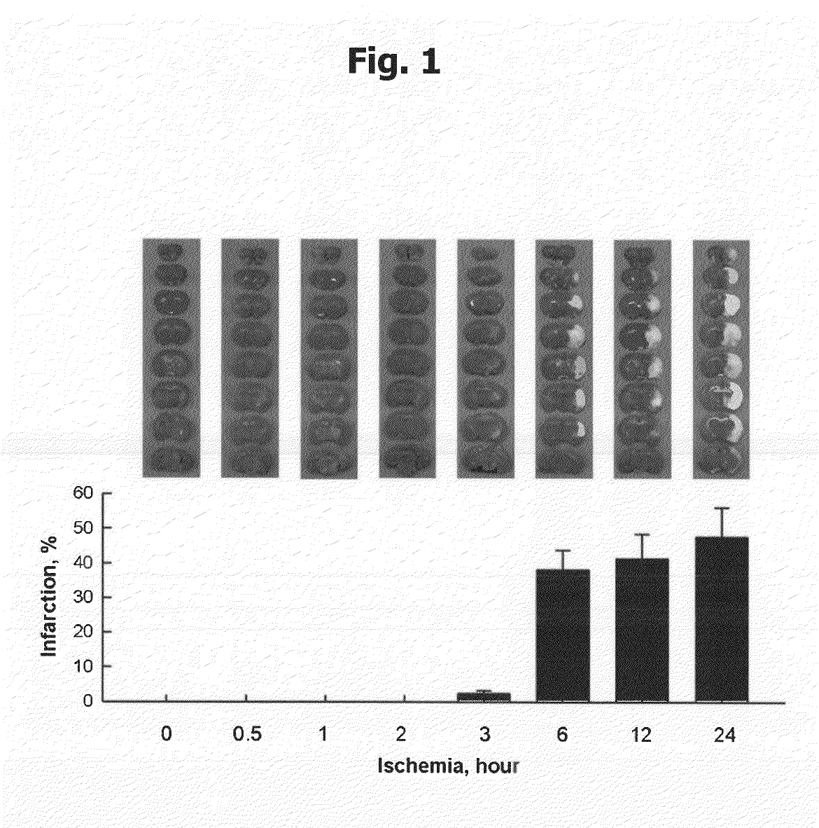

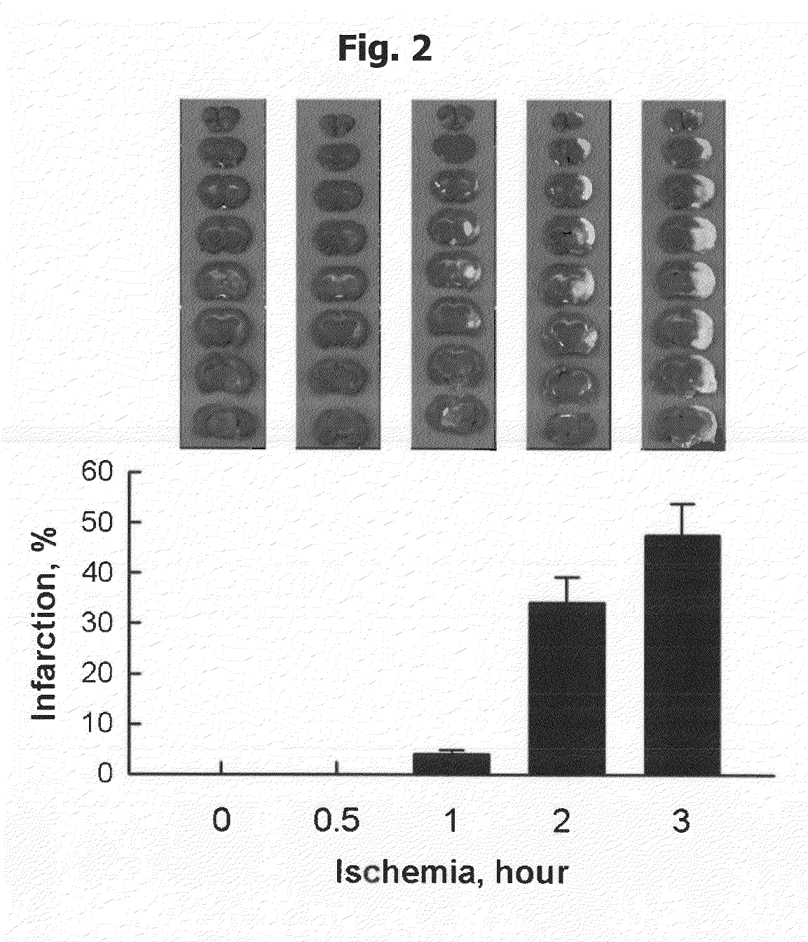

The infarction area was revealed by TTC staining after various durations (0.5-24 hours) of ischemia. Prominently visible infarctions were observed in the cortices, hippocampus and thalamus in the ipsilateral hemisphere after 6-hour occlusion, although a small (2.3%) lesions were observed in 3-hour ischemic brains (FIG. 1). Such brain infarctions were remarkably facilitated by 24 hours of reperfusion following 0.5-3-hour ischemia, exhibiting profound infarctions in 2- and 3-hour occlusion groups (FIG. 2). Only minimal (3.8%) injury was observed in the 1-hour occlusion group, while no visible changes were seen in the 0.5-hour occlusion group.

Differential Release of GFAP and MAP2 into the CSF and Blood

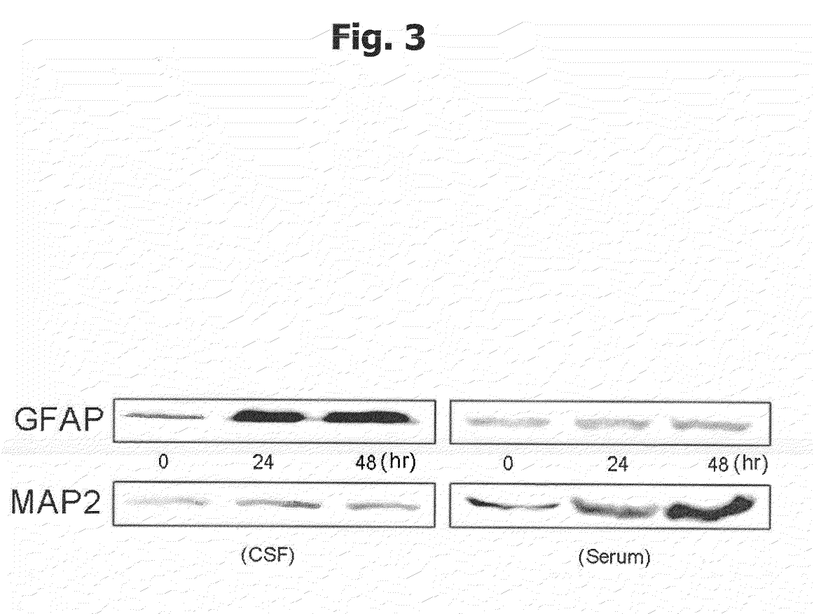

24 and 48 hours after 3-hour ischemia, the levels of GFAP and MAP2 in the CSF and serum were analyzed. The level of GFAP drastically increased in the CSF at both 24 and 48 hours, while it did not change in the serum up to 48 hours (FIG. 3). However, the level of MAP2 prominently increased in the serum and only slightly increased in the CSF.

Effect of the Degree of Infarction on GFAP and MAP2 Release into the Blood

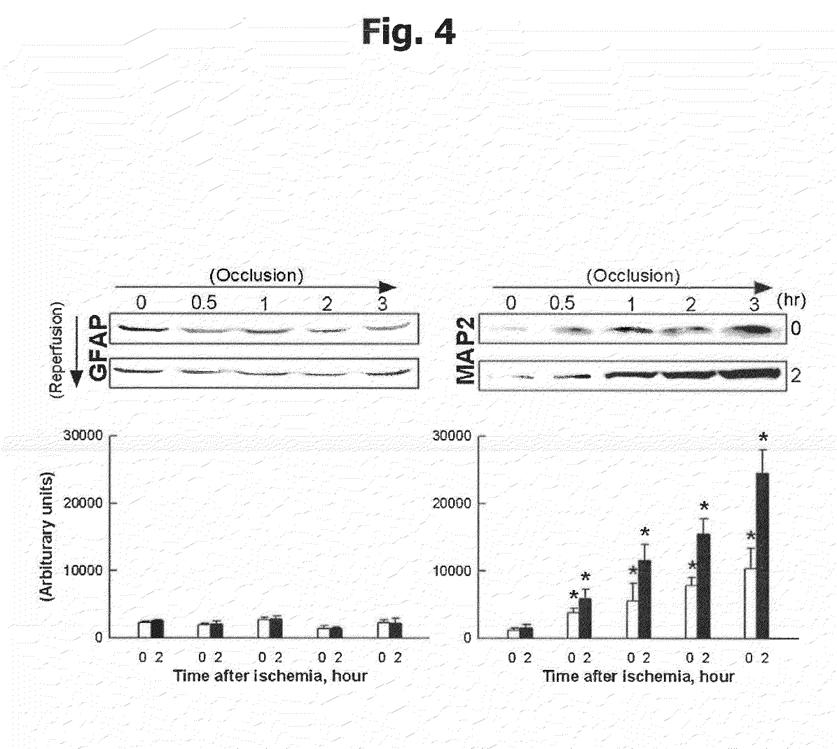

In order to analyze the effect of the severity of brain insult on the early release of MAP2 into the serum, the blood samples were collected immediately before (0 hour) or 2 hours after reperfusion following various durations (0.5-3 hours) of ischemia treatment. In contrast to a negligible ischemia and/or reperfusion-related change in GFAP release, MAP2 levels markedly increased in an ischemia duration-dependent manner, leading to a significant increase even in the 0.5-hour ischemic group (FIG. 4). Notably, the blood level of MAP2 was doubled by 2-hour reperfusion at longer durations of ischemia.

Time-Course of MAP2 Release into the Blood

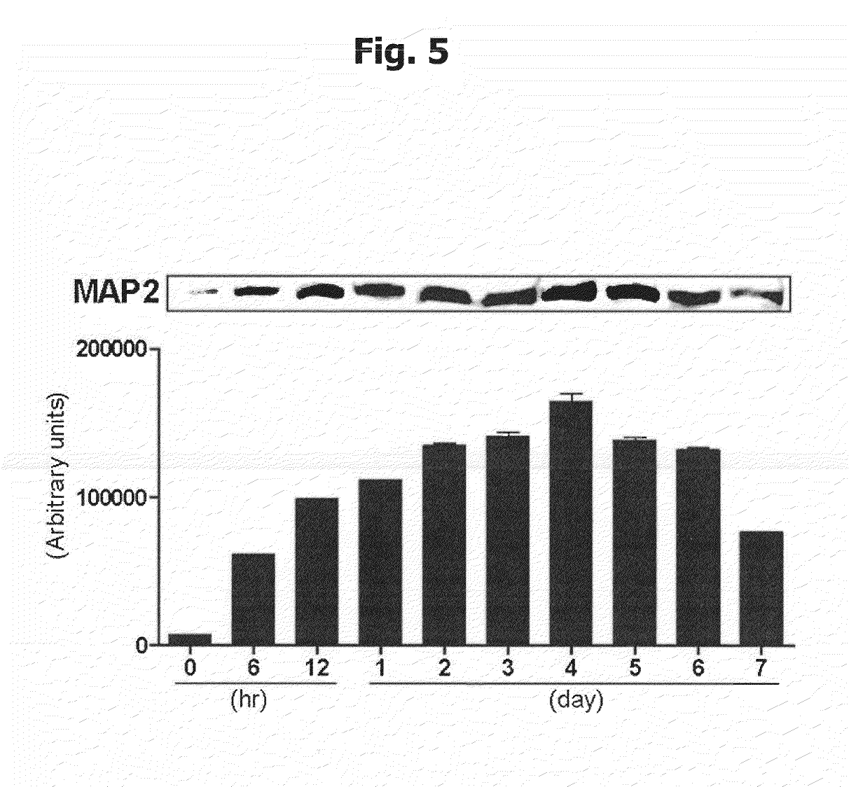

After 3 hours of ischemia followed by reperfusion, the release of MAP2 into the blood was markedly enhanced, exhibiting a peak between days 4 and 6 and rapidly decreasing thereafter (FIG. 5). The blood level of MAP2 increased 6 fold over the control as early as 6 hours after reperfusion, and reached a near-peak level in day 2.

### Comparison of MAP2 Between Brain and Serum

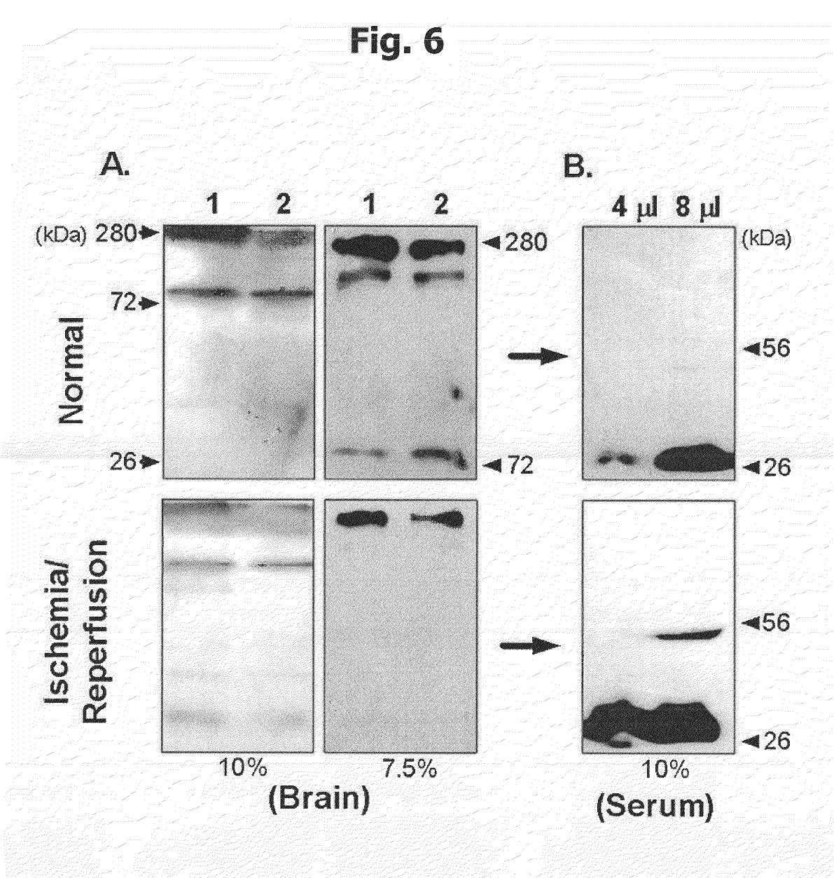

Next, MAP2 was identified in the ischemia/reperfused brain and serum. To determine what forms of MAP2 are released into the blood, serial Western blots were analyzed. In a normal brain, MAP2 exists at forms of high-molecular weight-MAP2 (HMW-MAP2) with molecular weight of ˜280 kDa and low-molecular weight-MAP2 (LMW-MAP2) with molecular weights close to 70 kDa (FIG. 6A). Interestingly, in an ischemia/reperfused brain, a lower part of HMW-MAP2 and LMW-MAP2 were disappeared, and a smaller form of MAP2 (˜50 kDa) was identified in the serum, which was not found in a normal rat serum (FIG. 6B). These results strongly suggest that MAP2 is released from the ischemia/reperfused brain into the blood at the early time points of tissue injury, large enough to be detectable.

### Discussion

Current stroke treatments are typically limited to no supportive care and secondary stroke prevention, resulting in only limited improvements in cognitive and motor function after the patient recovers from the stroke [27]. Only intravenous administration of t-PA has been effective in ameliorating the neurological deficits that arise from acute stroke [15]. However, it is recommended that t-PA should be administered within 3 hours following the onset of the stroke. As a result, this treatment only benefits less than 3% of ischemic stroke patients [28]. Moreover, an extremely careful treatment of t-PA under accurate monitoring of the status of the diseases is needed, because t-PA could disrupt blood vessels, leading to an acute fatal hemorrhage [16]. Since brain imaging techniques have a limited capacity to detect microlesions or progressive biochemical changes in neural cells at this early stage [1, 29], sensitive markers, in the CSF [30-32] or blood [2-4], derived from the neural tissue or dying cells could be helpful for monitoring the degree and progression of the brain damage.

MAPs comprise a class of cytoskeletal proteins that are essential for the functions of microtubules and are generally believed to serve as microtubule-connecting links to organelles, vesicles and other cytoskeletal elements [33]. MAP2 has also been used as a sensitive marker for brain damage in diverse disease models including seizures [34], ischemia and hypoxia [19-22, 35] and contusive brain injuries [36]. However, MAP2 was initially established as a marker that disappeared from the damaged neurons, which was detected immunohistochemically, and was believed to have very limited use as a clinical diagnostic marker. Therefore, we hypothesized that the disappearance of the MAP2 protein from neurons resulted from a flow of this potential marker into the CSF or blood.

The MCAO model described here has been commonly used as a standard protocol for the study of adult ischemic stroke as well as perinatal hypoxia/ischemia [37, 38]. In this model, the infarction volume had a strong correlation with not only the duration of ischemia, but also with the serum level of MAP2, which increased quite rapidly (FIG. 4). This increase in MAP2 may be related to its prompt loss in brain tissue following ischemic and contusive injuries [19, 36]. Moreover, a significant increase in the serum level of MAP2 was observed as early as 0.5-2-hour ischemic rats (FIG. 4) that did not have any visible infarction (FIG. 1). Moreover, 2-hour reperfusion further increased the MAP2 level in 0.5-hour ischemic rats (FIG. 4), in spite of lack of visible lesions after 24 hours (FIG. 2). In comparison, the serum level of GFAP did not change after 2 hour reperfusion even in the case of long-term (2-3 hours) ischemia (FIG. 4), which resulted in severe brain infarction and a drastic increase in the level of GFAP in the CSF 24 hours later (FIGS. 2 and 3). In fact, a significant increase in the blood level of GFAP was reported 2 to 3 days after a patient suffered an acute stroke [3,11], which may be due to the lag time between neuronal injury and responsible astrocytosis. Since it has been recommended that t-PA be administered within 3 hours of stroke onset [15], MAP2 may be a superior early blood marker than GFAP for detecting ischemic CNS injury.

It has been well documented that MAP2 is extremely sensitive to ischemia and may be an effective early marker of ischemia-induced neuronal damage [39, the present invention]. Intracellular calcium concentration increases during anoxia/ischemia [40], followed by a secondary phase of cellular calcium overload simultaneous with or slightly preceding the development of hypoxic-ischemic neuronal damage [41].

Thus, activation of calpain-induced proteolysis of MAP2 may result in the initial release of MAP2 into the body fluid [18]. It has also been suggested that the loss of MAP2 in association with excitotoxic brain damage stems from proteolytic degradation by calcium-activated proteases [34].

Hypoxia, which is associated with disorders such as stroke, and seizures disrupt BBB [24, 42, 43]. Alterations in endothelial cells, such as increased pinocytotic vesicles and derangement of tight junction proteins, may be responsible for the increased permeability of BBB, which results in the swelling of astrocyte end feet [24]. It is worth noting that the amount of MAP2 released into the blood was highly ischemia-duration dependent and was markedly facilitated by reperfusion (FIG. 4). Therefore, it is believed that MAP2 degraded by calcium-activated proteases under hypoxic conditions flow into the blood during BBB-disrupting ischemia, and more easily during reperfusion. This theory is consistent with previous results where BBB openings allowed macromolecules, such as S100B and α₂-macroglobulin, to be released [23, 24].

Interestingly, there were large differences between the CSF and serum levels of both the MAP2 and GFAP markers after neural injury, indicating different release mechanisms. Astrocyte-derived S100B and GFAP were predominantly observed in the CSF, where the protein levels were 40-50 times higher than in the serum [14,44,45, FIG. 2]. In contrast, NSE, a neuron-specific protein, had a 1:1 CSF/serum ratio [46]. Interestingly, MAP2 was much higher in the serum (6 to 14 times at 24-48 hours) than in the CSF (FIGS. 3 and 5), although the different release mechanisms of CNS proteins into the CSF and blood are still unknown.

In the present invention, we observed a significant increase in the serum level of MAP2 as early as 0.5 hour in ischemic animals that had no visible infarction. Also, a more prominent increase in MAP2 was achieved within 2 hours in 0.5-1-hour ischemia/reperfused rats, which later displayed no or minimal infarction. Notably, it is believed that increased shorter forms of MAP2 (−50 kDa), neither HMW-MAP2 (−280 kDa) nor LMW-MAP2 (−70 kDa), may be released into the blood after ischemia/reperfusion, probably via an activation of calpain-induced proteolysis of MAP2. These results indicate that early microlesions in neurons can be detected by analyzing the level of MAP2 in the blood, which precede the appearance of visible lesions. Therefore, we proposed that MAP2, which exhibits a high level in the blood, could be used as an early sensitive index of neurotoxic insult, providing patients with information on the severity and prognosis of brain damage for emergency or follow-up treatment.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Gilman S (1998) Imaging the brain. First of two parts. N Engl J Med 338: 812-820.
2. Wunderlich M T, Ebert A D, Kartz T, Goertler M, Jost S, et al. (1999) Early neurobehavioral outcome after stroke is related to release of neurobiochemical markers of brain damage. Stroke 30: 1190-1195.
3. Herrmann M, Vos P, Wunderlich M T, de Bruijn C H M M, Lamers K J B (2000) Release of glial tissue-specific proteins after acute stroke. A comparative analysis of serum concentrations of protein S-100B and glial fibrillary acidic protein. Stroke 31: 2670-2677.
4. Wunderlich M T, Wallesch C W, Goertler M (2004) Release of neurochemical markers of brain damage is related to the neurovascular status on admission and the site of arterial occlusion in acute ischemic stroke. J Neurol Sci 227: 49-53.
5. Zimmer D B, Cornwall E H, Landar A, Song W (1995) The S100 protein family: history, function, and expression. Brain Res Bull 37: 417-429.
6. Ilg E C, Schäfer B W, Heizmann C W (1996) Expression pattern of S100 calcium-binding proteins in human tumors. Int J Cancer 68: 325-332.
7. Lamers K J B, Vos P, Verbeek M M, Rosmalen F, van Geel W J, et al. (2003) Protein S-100B, neuron-specific enolase (NSE), myelin basic protein (MBP) and glial fibrillary acidic protein (GFAP) in cerebrospinal fluid (CSF) and blood of neurological patients. Brain Res Bull 61: 261-264.
8. Steiner J, Bernstein H G, Bogerts B, Gos T, Richter-Landsberg C, et al. (2008) S100B is expressed in, and released from, OLN-93 oligodendrocytes: influence of serum and glucose deprivation. Neuroscience 154: 496-503.
9. Cunningham R T, Watt M, Winder J, McKinstry S, Lawson J T, et al. (1996) Serum neuron-specific enolase as an indicator of stroke volume. Eur J Clin Invest 26: 298-303.
10. Vos P E, van Gils M, Beems T, Zimmerman C, Verbeek M M (2006) Increased GFAP and S100beta but not NSE serum levels after subarachnoid haemorrhage are associated with clinical severity. Eur J Neurol 13: 632-638.
11. Vissers J L M, Mersch M E C, Rosmalen C F, van Heumen M J M T, van Geel W J A, et al. (2006) Rapid immunoassay for the determination of glial fibrillary acidic protein (GFAP) in serum. Clin Chim Acta 366: 336-340.
12. Wunderlich M T, Wallesch C W, Goertler M (2006) Release of glial fibrillary acidic protein is related to the neurovascular status in acute ischemic stroke. Eur J Neurol 13: 1118-1123.
13. Kim J S, Yoon S S, Kim Y H, Ryu J S (1996) Serial measurement of interleukin-6, transforming growth factor-β, and S-100 protein in patients with acute stroke. Stroke 27: 1553-1557.
14. Foerch C, Wunderlich M T, Dvorak F, Humpich M, Kahles T, et al. (2007) Elevated serum S100B levels indicate a higher risk of hemorrhagic transformation after thrombolytic therapy in acute stroke. Stroke 38: 2491-2495.
15. The National Institute of Neurological Disorders and Stroke it-PA Stroke Study Group (1995) Tissue plasminogen activator for acute ischemic stroke. N Engl J Med 333: 1581-1587.
16. Zivin J A (1999) Thrombolytic stroke therapy: past, present, and future. Neurology 53: 14-19.
17. Tucker R P (1990) The roles of microtubule-associated proteins in brain morphogenesis:a review. Brain Res Brain Res Rev 15: 101-120.
18. Johnson G V W, Jope R S (1992) The role of microtubule-associated protein 2 (MAP-2) in neuronal growth, plasticity, and degeneration. J Neurosci Res 33: 505-512.
19. Kitagawa K, Matsumoto M, Ninobe M, Mikoshiba K, Hata R, et al. (1989) Microtubule-associated protein 2 as a sensitive marker for cerebral ischemic damage—immunohistochemical investigation of dendritic damage. Neuroscience 31: 401-411.
20. Zhang Z G, Bower L, Zhang R L, Chen S, Windham J P, et al. (1999) Three-dimensional measurement of cerebral microvascular plasma perfusion, glial fibrillary acidic protein and microtubule associated protein-2 immunoreactivity after embolic stroke in rats: a double fluorescent labeled laser scanning confocal microscopic study. Brain Res 844: 55-66.
21. Kitano H, Nishimura H, Tachibana H, Yoshikawa H, Matsuyama T (2004) ORP150 ameliorates ischemia/reperfusion injury from middle cerebral artery occlusion in mouse brain. Brain Res 1015: 122-128.
22. Pastori C, Regondi M C, Librizzi L, de Curtis M (2007) Early excitability changes in a novel acute model of transient focal ischemia and reperfusion in the in vitro isolated guinea pig brain. Exp Neurol 204: 95-105.
23. Cucullo L, Marchi N, Marroni M, Fazio V, Namura S, et al. (2003) Blood-brain barrier damage induces release of α₂-macroglobulin. Mol Cell Proteomics 2: 234-241.
24. Kaur C, Ling E A (2008) Blood brain barrier in hypoxic-ischemic conditions. Curr Neurovasc Res 5: 71-81.
25. Longa E Z, Weinstein P R, Carlson S, Cummins R (1989) Reversible middle cerebral artery occlusion without craniectomy in rats. Stroke 20: 84-91.
26. Chou J, Harvey B K, Chang C F, Shen H, Morales M, et al. (2006) Neurogenerative effects of BMP7 after stroke in rats. 3 Neurol Sci 240: 21-29.
27. Bitsch A, Horn C, Kemmling Y, Seipelt M, Hellenbrand U, et al. (2002) Serum tau protein level as a marker of axonal damage in acute ischemic stroke. Eur Neurol 47: 45-51.
28. Bliss T, Guzman R, Daadi M, Steinberg G K (2007) Cell transplantation therapy for stroke. Stroke 38: 817-826.
29. Kane I, Whiteley W N, Sandercock P A G, Wardlaw J M (2008) Availability of CT and MR for assessing patients with acute stroke. Cerebrovasc Dis 25: 375-377.
30. Rosengren L E, Wikkelsø C, Hagberg L (1994) A sensitive ELISA for glial fibrillary acidic protein: application in CSF of adults. J Neurosci Mehods 51: 197-204.
31. Anand N, Stead L G (2005) Neuron-specific enolase as a marker for acute ischemic stroke: A systematic review. Cerebrovasc Dis 20: 213-219.
32. Tanaka Y, Marumo T, Omura T, Yoshida S (2008) Relationship between cerebrospinal and peripheral S100B levels after focal cerebral ischemia in rats. Neurosci Lett 436: 40-43.
33. Shepherd G M (1994) Neuron and glia. In: Shepherd G M, editor. Neurobiology. New York: Oxford University Press. pp. 36-64.
34. Ballough G P H, Martin L J, Cann F J, Graham J S, Smith C D, et al. (1995) Microtubule-associated protein 2 (MAP-2): a sensitive marker of seizure-related brain damage. J Neurosci Methods 61: 23-32.
35. Yushmanov V E, Kharlamov A, Simplaceanu E, Williams D S, Jones S C (2006) Differences between arterial occlusive and cortical photothrombosis stroke models with magnetic resonance imaging and microtubule-associated protein-2 immunoreactivity. Magn Reson Imaging 24: 1087-1093.
36. Saatman K E, Feeko K J, Pape R L, Raghupathi R (2006) Differential behavioral and histopathological responses to graded cortical impact injury in mice. J Neurotrauma 23: 1241-1253.
37. Vannucci R C, Connor J R, Mauger D T, Palmer C, Smith M B, et al. (1999) Rat model of perinatal hypoxic-ischemic brain damage. J Neurosci Res 55: 158-163.
38. O'Donell S L, Frederick T J, Krady J K, Vannucci S J, Wood T L (2002) IGF-I and microglia/macrophage proliferation in the ischemic mouse brain. Glia 39: 85-97.
39. Yoshimi K, Takeda M, Nishimura T, Kudo T, Nakamura Y, et al. (1991) An immunohistochemical study of MAP2 and clathrin in gerbil hippocampus after cerebral ischemia. Brain Res 560: 149-158.
40. Puka-Sundvall M, Hagberg H, Andine P (1994) Changes in extracellular calcium concentrations in the immature rat cerebral cortex during anoxia is not influenced by MK-801. Dev Brain Res 77: 146-150.
41. Stein D T, Vannucci R C (1988) Calcium accumulation during evolution of hypoxic-ischemic brain damage in the immature rat. J Cereb Blood Flow Metab 8: 834-842.
42. Choi E K, Park D, Yon J M, Hur G H, Ha Y C, et al. (2004) Protection by sustained release of physostigmine and procyclidine of soman poisoning in rats. Eur J Pharmacol 505: 83-91.
43. Park D, Jeon J H, Shin S, Jang J Y, Choi B, et al. (2008) Debilitating stresses do not increase blood-brain barrier permeability: lack of the involvement of corticosteroids. Environ Toxicol Pharmacol 26: 30-37.
44. Kanner A A, Marchi N, Fazio V, Mayberg M R, Koltz M T, et al. (2003) Serum S100beta: a noninvasive marker of blood-brain barrier function and brain lesions. Cancer 97: 2806-2813.
45. Marchi N, Rasmussen P, Kapural M, Fazio V, Kight K, et al. (2003). Peripheral markers of brain damage and blood-brain barrier dysfunction. Restor Neurol Neurosci 21: 109-121.
46. Reiber H (2003) Proteins in cerebrospinal fluid and blood: barriers. CSF flow rate and source-related dynamics. Restor Neurol Nuerosci 21: 79-96.

What is claimed is:

1. A method for detecting neuronal injury in a mammalian subject, comprising:
(a) providing a biological sample from the mammalian subject; and
(b) detecting the level of a MAP2 (microtubule-associated protein 2) protein or a MAP2 nucleic acid in the biological sample, relative to the level of the MAP2 protein or the MAP2 nucleic acid in a control sample from a normal mammalian, wherein an increased level of the MAP2 protein or the MAP2 nucleic acid in the biological sample compared to the control sample indicates that the mammalian has the neuronal injury.

2. The method according to claim 1, wherein the biological sample is blood, serum or plasma.

3. The method according to claim 1, wherein the detection of the step (b) is carried out by analyzing the level of the MAP2 protein.

4. The method according to claim 3, wherein the analysis of the level of the MAP2 protein is carried out by an immunoassay.

5. The method according to claim 1, wherein the neuronal injury is a neurological insult or a neurodegenerative disorder.

6. The method according to claim 1, wherein the neuronal injury is an ischemic neurological insult.

7. The method according to claim 5, wherein the detection of the step (b) is carried out within 10-60 min of the neurological insult.

8. The method according to claim 5, wherein the detection of the step (b) is carried out before the appearance of visible lesions in neuronal tissues.

9. The method according to claim 8, wherein the detection of the step (b) is carried out within 10-60 min of the neurological insult.
